(19) United States
(12) Patent Application Publication
(10) Pub. No.: US 2020/0022924 A1
(43) Pub. Date: Jan. 23, 2020

(54) Cannabidiol (CBD) Formulation Including Caprylic Acid

(71) Applicants: Cody D. Freeze, Pratt, KS (US); Allison N. Freeze, Pratt, KS (US)

(72) Inventors: Cody D. Freeze, Pratt, KS (US); Allison N. Freeze, Pratt, KS (US)

(21) Appl. No.: 16/040,113

(22) Filed: Jul. 19, 2018

(51) Int. Cl. A61K 31/05; A61K 9/00; A61K 36/185; A61K 47/24

(52) U.S. Cl. CPC A61K 31/05; A61K 9/006; A61K 31/20; A61K 47/24; A61K 2300/00; A61K 36/185

(57) ABSTRACT

A cannabinoid composition for enabling nearly simultaneous sublingual delivery of at least one cannabinoid, and an anti-fungal agent to the digestive tract of a subject. The composition includes cannabidiol, caprylic acid, lecithin, and at least one terpene. The ratio of cannabidiol to caprylic acid is between 10:1 and 25:1 on a weight to weight (w:w) basis. Preferably, the lecithin is liquid soy lecithin, the cannabidiol is an isolate having at least 95% purity, and the at least one terpene is engineered to have a therapeutic entourage effect in combination with the at least one cannabinoid in vivo.

### FIELD OF THE INVENTION

The present invention relates to cannabinoid formulations, including cannabidiol (CBD) formulations, more particularly the invention relates to sublingual delivery of CBD formulations.

### BACKGROUND OF THE INVENTION

Transdermal delivery of bio-active compounds have developed for human and animal consumption and therapeutic use since the beginning of recorded history. In ancient times herbs were gathered, compressed into a poultice and applied to the skin to treat wounds, pain and other maladies.

A subset of transdermal delivery methodologies includes sublingual delivery. The mucosal membrane and epithelium under the tongue is adept at absorbing nutrients and biologically active components. Moisture and mechanical movement in the oral cavity, and sub-lingual capillary density may enhance delivery of such bio-active components.

Sublingual administration has certain advantages over typical oral administration of bio-active components. During oral administration, many bio-active components may be effected by digestive enzymes of the gastrointestinal lumen, gut wall enzymes, bacterial enzymes and hepatic enzymes. The hepatic enzymes include many oxidative species that degrade cannabinoids including monoamine oxidase and monooxygenase. These and other enzymes are thought to be at least partially responsible for oxidizing, or otherwise transforming orally delivered classical cannabinoids, including cannabidiol prior to circulation throughout the bloodstream. Research indicates that up to 97% of orally ingested cannabinoids fail to circulate in the blood stream of a subject.

Sublingual delivery avoids enzymatic action of the gastrointestinal lumen, as well as first pass metabolism in the liver. Sublingual delivery thus improves the bioavailability and effective duration of cannabinoid activity in the body.

What is desired is a cannabinoid formulation including a non-toxic excipient that improves shelf stability, bioavailability and efficacy of the bioactive compounds.

### SUMMARY OF THE INVENTION

The present invention includes a cannabinoid composition The product consists of isolated capra fatty acids having between six to twelve carbon atoms. Preferably the present invention includes predominately caprylic acid medium chain triglycerides (caMCT) having C8 carbon atoms. In an alternate embodiment, the present invention includes solely isolated caprylic acid medium chain triglycerides (caMCT) having C8 carbon atoms, though there may be traces of other medium chain triglercides in insufficient concentrations to have any detectable bioactive influence in the formulated product of the present invention. It can be appreciated that various ratios of capra fatty acids can be designed and combined in various ratios to achieve the goals of the present invention.

The present invention further includes at least one cannabinoid. Preferably, the cannabinoid is an isolate having at least 90% purity, and more preferably having at least 95% purity. In a preferred embodiment, the cannabinoid is cannabidiol (CBD) in the form of an isolate powder derived from industrial hemp. It can be appreciated that a myriad of cannabinoids can be used in accordance with the present invention to achieve desirable biological effects in humans and other mammals.

The present invention includes an additional lypophilic excipient, carrier, or emulsification agent. Preferably the excipient, carrier, or emulsification agent is lecithin. In a preferred embodiment, the lecithin is derived from sunflower, and is in a liquid form. Lecithin has bio-activity that complements the bioactivity of the combination of CBD and caprylic acid.

A preferred standardized product in accordance with the present invention is packaged in a 30 mL vial. The container includes a total 500 mg CBD. Containers are 30 mL glass vial with dropper. In one embodiment of the invention, there are no terpenes added and this is termed a “plain” CBD oil preparation.

In another embodiment, terpenes and other cannabinoids are added to mimic a whole plant extract of cannabis sativa L, such as hemp or marijuana. More preferably, the terepene and cannabinoid concentrations do not exceed the ratios of terpenes and cannabinoids naturally found in medicinal varieties of the hemp or marijuana plant.

The ratios of terpenes and cannabinoids can be individualized on a patient to patient basis and adapted to target each of the following common cannabinoid treatment areas: pain and inflammation, focus and energy, mood and well being, gastrointestinal issues, and sleep and relaxation.

Terpenes including beta myrcene, limonene, beta caryophyllene, alpha humulene, alpha pinene, borneol, linalool, eucalyptol, nerolidol, phellandrene, phytol, pulegone, bergamotene, farmesene, D3-carene, elemene, fenchol, aromadendrene, bisabolene, and many more that have been documented as being found in Cannabis sativa L.

In yet another embodiment of this invention, the cannabinoids used include a combination of tetrandyrocannabinol (THC) and cannabidiol (CBD). In another embodiment, the cannabinoids are selected from the group consisting of: CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), THCVA (Tetrahydrocanabivarinic acid), CBDVA (C annabidivarinic acid), CBCVA (Cannabichromevarinic acid), the non-acid forms of the foregoing, and combinations thereof.

It can be appreciated that the term cannabiniod, whether relating to a specific cannabinoid, or generally referencing a group of cannabinoids, includes both the acid and non-acid forms of the molecule as well as isoforms thereof. Cannabinoids can be phytocannabinoids, synthetic cannabinoids, or endocannabinoids. Synthetic cannabinoids include those derived from engineered micro organisms such as yeast. Cannabinoids should be broadly construed to include precursors of particular cannabinoids, or oxidized products thereof including CBN (Cannabinol), and includes all cannabinoids found in the family of plants known as Cannabacae such as those found in the species Cannabis sativa L.

In an alternate embodiment of the invention, the cannabinoids in the inventive formulation can be a whole plant extract of Cannabis sativa L. Preferably this whole plant extract has above a fifty percent cannabinoid content.

Caprylic acid is a saturated fatty acid characterized by the chemical formula CH₃(CH₂)₆COOH, including isoforms thereof. Caprylic acid is a well-known antimicrobial found naturally in the milk of various mammals and as a constituent of coconut oil and palm kernel oil. It is minimally soluble in water. Caprylic acid is often used as an algaecide, bactericide, and fungicide that is non-toxic to humans and other mammals.

One benefit of including caprylic acid in the formulation of the present invention is to function as a preservative, enhancing shelf life and safety of the products of the present invention by inhibiting microbial contamination of the formulated product.

Caprylic acid is an anti-fungal agent. It also works synergistically with CBD to reduce inflammation in vivo. While the mechanism of action is not fully understood, it is believed that the short chain length of the Caprylic acid molecule (fatty acid) enables penetration via the cell walls of candida yeast in the gastrointestinal tract, causing cell death of the candida yeast. Accordingly, the caprylic acid enables delivery of the CBD sublingually, swallowing the non-absorbed portion of the formulation enables caprylic acid to enter the gastrointestinal tract to reduce candida in the gut.

Since candida yeast is implicated as one cause of chronic inflammation in humans, reducing the population of candida in the gut is seen as beneficial in aiding the reduction in chronic inflammation. The use of CBD, an adaptogen, triggers various cannabinoid receptors in the body, and influences the body's immune response, including any inflammatory response. This, when combined with the nearly simultaneous reduction of pathogenic yeast in the digestive tract, can achieve many desirable effects.

### DETAILED DESCRIPTION

The product consists of caprylic acid medium chain triglycerides (caMCT), CBD isolate derived from industrial hemp, and sunflower liquid lecithin. These are combined in accordance with the methods of the present invention in a standardized formulation.

In one embodiment, the standardized formulation is packaged to include 500 mg of CBD per 30 mL container. Containers are preferably a 30 mL glass vial with a dropper for sublingual delivery of the formation to a patient or subject.

In one embodiment of the invention, terpenes are added to the formulation to target each of the following common cannabinoid treatment areas: pain and inflammation, focus and energy, mood and well being, gastrointestinal issues, and sleep and relaxation. Each profile has a specific set amount of terpene blends, and this profile is specific to the intent of the supplement.

The invention preferably includes the functional excipients caMCT (caprylic acid), sunflower liquid lecithin, and phyto-terpenes. It can be appreciated that other types of lecithin can be substituted for the sunflower lecithin such as soy-derived lecithin, however sunflower lecithin is preferred because it is hypo-allergenic.

These excipients are chosen as being multi-functional. The excipients are lipophylic to simplify manufacturing a homogeneous and shelf-stable product. The lecithin enables delivery across the blood brain barrier because lecithin closely resembles the composition of lipids in the brain. The caprylic acid optimizes sublingual, transdermal, and transmucosal absorption of cannabinoids in humans. Additionally the caprylic acid as additional therapeutic benefits such as reducing pathogenic fungal growth in vivo. This combination of excipients and actives cooperate to minimize microbial growth to enhance safety and shelf life of the product of the present invention. Adding particular terpenes can enhance this cooperation of excipients and actives while enabling the efficacy of the cannabinoid(s).

The physiochemical characteristics of caprylic acid are optimal to yield a suitable viscosity for the formulation of the present invention. The pH of caprylic acid helps preserve the formulation, improving shelf life. The melting point is below room temperature to stabilize the viscosity at above the melting point. The details of the physiochemical properties of caprylic acid are expressed in Table 1, below.

TABLE 1
Physicohemical Characteristics of Caprylic acid

| | |
|---|---|
| Boiling point | 237° C. |
| | (1013 hPa) |
| Density | 0.91 g/cm3 |
| | (20° C.) |
| Explosion limit | 1% (V) |
| Flash point | 130° C. |
| Ignition temperature | >300° C. |
| Melting Point | 16° C. |
| pH value | 4 (0.2 g/l, H₂O, |
| | 20° C.) |
| Vapor pressure | 0.05 hPa |
| | (20° C.) |
| Aqueous Solubility | 0.68 g/l |

CBD is lypophylic as are the excipients of the present invention. The formulation of the present invention is designed to optimize oral absorption under the tongue and across the oral mucosa with the combination of lipids and lecithin. In order to achieve geometric addition and disbursement/emulsification, agitation via stir plate with light heat is utilized in the manufacturing of the formulation.

Lecithin is very common in the brain. Since the body recognizes the need for lecithin transport into and around the central nervous system, the addition of lecithin is an important component to the formulation because it enhances the flow of cannabinoid(s) between the bloodstream across the blood-brain barrier.

During manufacturing the mixture of base ingredients are heated. After heat is applied to the mixture of base ingredients (CBD powder+lecithin+caMCT), the product is mixed, emulsified, and transferred and packaged into individual oral 30 mL dropper bottles.

Post emulsification, terpenes are added in accordance with one embodiment of the invention. The addition and presence of terpenes in specific blends are aimed to supplement the human endocannabinoid system in ways to maximize synergism of parts to maximize the entourage effect, which is well-known in cannabinoid science. Preferably, volatile and other terpenes are added after the emulsification step, and after the product is cooled to reduce volitizatization of the terpenes.

Simply stated, terpenes assist the CBD molecule's adaptogenic activation of the human endocannabinoid system. The functionality of any terpene blend, or isolated terpene is engineered to be specific to each subject, or patient. It may be also specific to the needs of patients, the medications the patients take, and other factors. An example of a batch manufacturing process is expressed in Example#1.

### EXAMPLE #1

### Batch Preparation Steps

Providing 50mL sunflower liquid lecithin.
Providing 14.7 grains cannabidiol isolate powder from industrial hemp having at least 90% purity.
Providing 882 mL caMCT.
Mixing and heating the base ingredients set forth above to approximately 65 degrees Celsius with a magnetic stirrer until the mixture is emulsified, having a homogeneous in color and consistency.
Cooling to room temperature and adding individual or blended Terpenes. The terpene blends are engineered to the form/function for particular products and intended patients or subjects.
Packaging in a dropper bottle equipped with a dropper capable of sublingual administration.

### Example #2

### Administration Method:

A subject or patient holds ½ to 1 dropperful of the product of the invention sublingually for 30 seconds twice daily, or about every 12 hours. What is not absorbed by being held under tongue 30 seconds is subsequently to be swallowed to enable anti-fungal activity of residual excipient (caMCT). It can be appreciated that bioactivity of the cannabinoid extends for up to 12 hours due to the sublingual delivery method, which avoids first-pass metabolism associated with typical oral delivery through the gastrointestinal tract.

While the present invention is described in terms of various exemplary embodiments, the scope of the present invention is particularly defined by the appended claims. The claims use a weight to weight basis to define various ratios and this is expressed as “w:w”. Further the term “terpene” includes plant terpenes such as monoterpenes, diterpenes, triterpenes, sesqiterpenes, and other plant-derived terpenes having an array of possible isoprene units.

1. A cannabinoid composition for enabling sublingual delivery of at least one cannabinoid and delivery of an anti-fungal agent to the digestive tract of a subject, comprising:
cannabidiol;
caprylic acid;
lecithin; and
the ratio of the cannabidiol to the caprylic acid is 1:54.6 on a w:w basis.

2. (canceled)

3. (canceled)

4. The cannabinoid composition as set forth in claim 1, wherein the lecithin is sunflower lecithin.

5. The cannabinoid composition as set forth in claim 1, wherein the cannabinoid composition is a standardized product having between 16-17 mg cannabidiol per mL of the cannabinoid composition.

6. The cannabinoid composition as set forth in claim 1, further comprising: terpenes derived from Cannabis sativa L.

7. A cannabinoid composition comprising:
cannabidiol; and
a preservative having the chemical formula CH₃(CH₂)₆COOH,
the ratio of the cannabidiol the preservative is between 1:15 to 2:1 on a w:w basis.

8. The cannabinoid composition as set forth in claim 7, wherein the cannabidiol has at least a 90% purity.

9. (canceled)

10. The cannabinoid composition as set forth in claim 7 further comprising lecithin.

11. The cannabinoid composition as set forth in claim 7, wherein the lecithin, preservative and cannabidiol are emulsified.

12. (canceled)

13. The cannabinoid composition as set forth in claim 7, further comprising a terpene derived from Cannabis sativa L, and wherein the terpene is added after emulsification.

14.-16. (canceled)

17. The cannabinoid composition as set forth in claim 10, wherein the lecithin, preservative and cannabinoid are emulsified.
